(19) United States
(12) Patent Application Publication
(10) Pub. No.: US 2025/0362250 A1
(43) Pub. Date: Nov. 27, 2025

(54) RADIOGRAPHY APPARATUS, TEMPERATURE CONTROL METHOD OF RADIOGRAPHY APPARATUS, AND TEMPERATURE CONTROL PROGRAM

(71) Applicant: FUJIFILM Corporation, Tokyo (JP)

(72) Inventors: Yukio KUMAGAI, Tokyo (JP); Tatsuhiko HAYASHI, Tokyo (JP); Masafumi ONOUCHI, Tokyo (JP)

(73) Assignee: FUJIFILM Corporation, Tokyo (JP)

(21) Appl. No.: 19/216,797

(22) Filed: May 23, 2025

(30) Foreign Application Priority Data
May 27, 2024 (JP) 2024-085678

(51) Int. Cl. G01N 23/046

(52) U.S. Cl. CPC G01N 23/046; G01N 2223/101; G01N 2223/3103; G01N 2223/3303

(57) ABSTRACT

A radiography apparatus includes a thermally conductive rotation plate that rotates about a rotation axis and to which a radiation source is fixed, a radiation detector that includes a thermally conductive housing and is fixed to the rotation plate with a gap partially present between the radiation detector and the rotation plate at a position opposite to the radiation source across the rotation axis of the rotation plate, and a heat dissipation mechanism that is disposed in the gap and has a thermally conductive material for dissipating heat of the radiation detector to the rotation plate, in which the thermally conductive material is moved to a position at which the radiation detector and the rotation plate are caused to be thermally conductive from a non-contact position with the rotation plate or the radiation detector in a situation where a temperature of the radiation detector rises.

### CROSS-REFERENCE TO RELATED APPLICATION

The present application claims priority from Japanese Patent Application No. 2024-085678, filed on May 27, 2024, the entire disclosure of which is incorporated herein by reference.

### BACKGROUND

### Technical Field

The present disclosure relates to a radiography apparatus, a temperature control method of a radiography apparatus, and a temperature control program.

### Description of the Related Art

In recent years, a photon counting computed tomography (PCCT) apparatus that is a radiography apparatus equipped with a photon counting detector has been known. Unlike a charge integration detector employed in a computed tomography (CT) apparatus in the related art, the photon counting detector can count photons of incident radiation. Since the PCCT apparatus can measure energy for each photon, more information can be obtained compared to the CT apparatus in the related art.

In the PCCT apparatus, incident photons are converted into charges in a semiconductor layer, and the photon counting is performed by a photon counting circuit counting the converted charges. Such the photon counting detector generates heat due to the counting of photons. Since the characteristics of the photon counting circuit change due to a temperature change caused by heat generation, various methods for keeping a temperature of the detector constant have been proposed.

For example, JP2024-037489A proposes a method for keeping a temperature of a detector constant by forming an openable and closable opening in a housing of the detector, acquiring information related to the heat generation amount, such as a temperature of the detector, and selectively opening and closing the opening based on the acquired information.

By the way, in a photon counting detector, the heat generation of a circuit provided in the detector, such as a photon counting circuit, is more remarkable than that of the detector of the related art. Therefore, in a case where only the method described in JP2024-037489A is used, there is a possibility that cooling of the detector is insufficient.

### SUMMARY OF THE INVENTION

The present disclosure has been made in view of the above circumstances, and an object of the present disclosure is to achieve temperature stabilization of a detector by further cooling the detector.

A radiography apparatus according to the present disclosure includes
a thermally conductive rotation plate that rotates about a rotation axis and to which a radiation source is fixed,
a radiation detector that includes a thermally conductive housing and is fixed to the rotation plate with a gap partially present between the radiation detector and the rotation plate at a position opposite to the radiation source across the rotation axis of the rotation plate, and
a heat dissipation mechanism that is disposed in the gap and has a thermally conductive material for dissipating heat of the radiation detector to the rotation plate,
in which the thermally conductive material is moved to a position at which the radiation detector and the rotation plate are caused to be thermally conductive from a non-contact position with the rotation plate or the radiation detector in a situation where a temperature of the radiation detector rises.

In the radiography apparatus according to the present disclosure,
the thermally conductive material may consist of a first member and a second member, each having a sliding surface that is inclined with respect to surfaces of the rotation plate and the radiation detector facing each other, and
the first member may be fixed to the rotation plate or the radiation detector in the gap, the second member may be located at a position away from the rotation plate or the radiation detector at an initial position, and the second member may be moved to an operation position at which the second member comes into contact with the radiation detector or the rotation plate while sliding on the first member to maintain contact with the first member in the situation where the temperature of the radiation detector rises.

In addition, in the radiography apparatus according to the present disclosure,
the heat dissipation mechanism may have a spring that is disposed along a radial direction of the rotation plate, of which one end is fixed to the rotation plate or the radiation detector and the other end located on a radially outer side of the rotation plate from the one end is fixed to the second member, and
a spring constant of the spring may be determined based on an angular velocity in a case where the rotation plate rotates, a position of the second member in the radial direction of the rotation plate, and a weight of the second member.

Further, in the radiography apparatus according to the present disclosure,
the heat dissipation mechanism may have a driving unit including a motor and a ball screw of which one end is fixed to the motor and the other end is screwed to the second member, and
the driving unit may move the second member from the initial position to the operation position and move the second member from the operation position to the initial position by driving the motor based on information related to a heat generation amount of the radiation detector.

Furthermore, in the radiography apparatus according to the present disclosure,
the information related to the heat generation amount may be a temperature measured by a temperature sensor provided in the radiation detector.

Moreover, in the radiography apparatus according to the present disclosure,
the information related to the heat generation amount may be information based on a tube current applied to the radiation source.

Additionally, in the radiography apparatus according to the present disclosure,
the thermally conductive material may be divided into a plurality of small members in a radial shape with respect to the rotation axis, and
the driving unit may change positions and the number of the small members that thermally conduct the radiation detector and the rotation plate according to at least one of a position at which the temperature in the radiation detector rises or the heat generation amount.

A temperature control method of a radiography apparatus according to the present disclosure includes
a thermally conductive rotation plate that rotates about a rotation axis and to which a radiation source is fixed,
a radiation detector that includes a thermally conductive housing and is fixed to the rotation plate with a gap partially present between the radiation detector and the rotation plate at a position opposite to the radiation source across the rotation axis of the rotation plate, and
a heat dissipation mechanism that is disposed in the gap and has a thermally conductive material for dissipating heat of the radiation detector to the rotation plate, and a driving unit that moves the thermally conductive material to a position at which the radiation detector and the rotation plate are caused to be thermally conductive from a non-contact position with the rotation plate or the radiation detector in a situation where a temperature of the radiation detector rises, the method comprising:
acquiring information related to a heat generation amount of the radiation detector by a computer; and
controlling driving of the thermally conductive material by the driving unit based on the information related to the heat generation amount.

A temperature control program according to the present disclosure, which causes a computer to execute a temperature control method in a radiography apparatus including
a thermally conductive rotation plate that rotates about a rotation axis and to which a radiation source is fixed,
a radiation detector that includes a thermally conductive housing and is fixed to the rotation plate with a gap partially present between the radiation detector and the rotation plate at a position opposite to the radiation source across the rotation axis of the rotation plate, and
a heat dissipation mechanism that is disposed in the gap and has a thermally conductive material for dissipating heat of the radiation detector to the rotation plate, and a driving unit that moves the thermally conductive material to a position at which the radiation detector and the rotation plate are caused to be thermally conductive from a non-contact position with the rotation plate or the radiation detector in a situation where a temperature of the radiation detector rises, the program causing a computer to execute a process comprising:
acquiring information related to a heat generation amount of the radiation detector; and
controlling driving of the thermally conductive material by the driving unit based on the information related to the heat generation amount.

According to the present disclosure, in a case of setting an imaging range, feature points can be appropriately detected according to a situation of a subject.

### BRIEF DESCRIPTION OF THE DRAWINGS

FIG. 1 is a diagram schematically showing a configuration of a CT apparatus that is an example of a radiography apparatus according to a first embodiment of the present disclosure.

FIG. 2 is a perspective view schematically showing a configuration of a detector.

FIG. 3 is an enlarged view of a portion of a detector in the CT apparatus according to the first embodiment that is attached to a rotation plate.

FIG. 4 is a cross-sectional view taken along line I-I of FIG. 3 (initial position of second member).

FIG. 5 is a cross-sectional view taken along the line I-I of FIG. 3 (operation position of second member).

FIG. 6 is a view showing a configuration of a heat dissipation mechanism in a CT apparatus according to a second embodiment.

FIG. 7 is a cross-sectional view taken along line II-II of FIG. 6 (initial position of second member).

FIG. 8 is a cross-sectional view taken along the line II-II of FIG. 6 (operation position of second member).

FIG. 9 is a flowchart showing processing performed in the second embodiment.

FIG. 10 is a flowchart showing processing performed in a third embodiment.

FIG. 11 is a view showing a configuration of a heat dissipation mechanism in a CT apparatus according to a fourth embodiment.

FIG. 12 is a view showing an operation of the heat dissipation mechanism in the fourth embodiment.

FIG. 13 is a view showing the operation of the heat dissipation mechanism in the fourth embodiment.

FIG. 14 is a view showing the operation of the heat dissipation mechanism in the fourth embodiment.

### DETAILED DESCRIPTION

Hereinafter, a description of embodiments of the present disclosure will be made with reference to the accompanying drawings. A CT apparatus according to a first embodiment is a PCCT apparatus that detects radiation emitted from a radiation source and generates a radiation image based on an electrical signal corresponding to the number of photons of the radiation. In the present embodiment, a case where the radiation is X-rays will be described as an example.

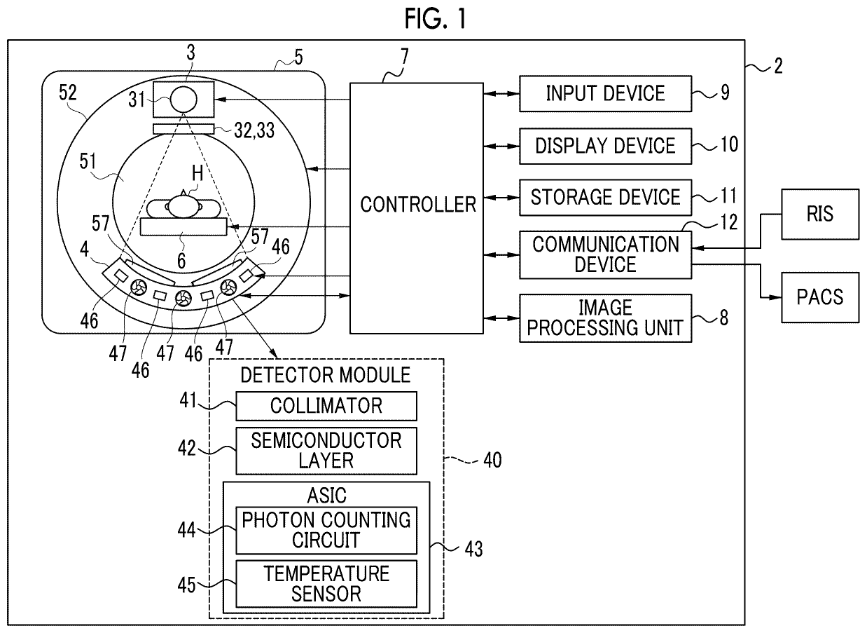

FIG. 1 is a diagram schematically showing a configuration of a CT apparatus that is an example of the radiography apparatus according to the first embodiment. A CT apparatus 2 includes an X-ray source 3, an X-ray detector 4, a gantry 5, an examination table 6, a controller 7, and an image processing unit 8. A circular opening portion 51 for disposing the examination table 6 on which a subject H is placed is provided at a center of the gantry 5. In addition, the gantry 5 is provided with a rotation plate 52 in which the X-ray source 3 and the X-ray detector 4 (hereinafter, simply referred to as the detector 4) are fixed at positions to face each other, and a drive mechanism (not shown) for rotating the rotation plate 52. In addition, in the first embodiment, a heat dissipation mechanism 57 is provided between the rotation plate 52 and the detector 4.

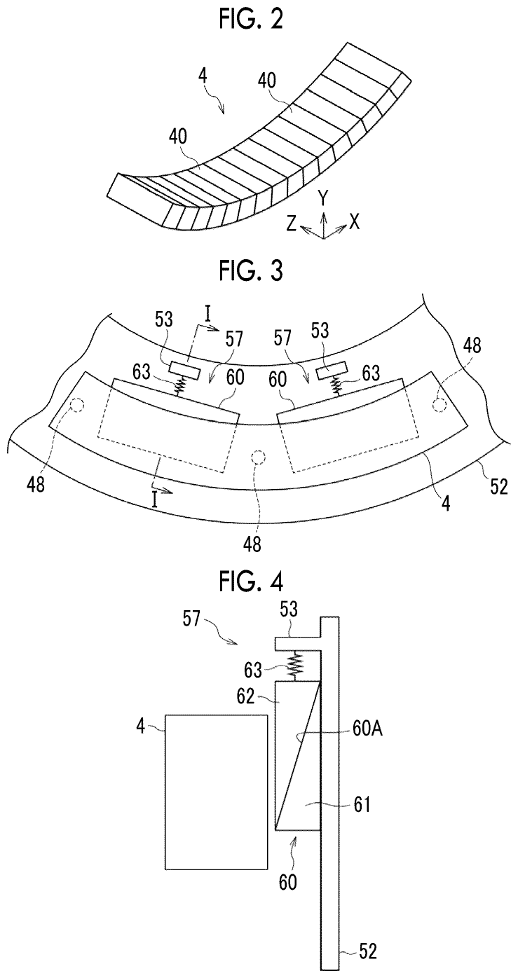

Hereinafter, in the present disclosure, a circumferential direction of the opening portion 51 is referred to as an X direction, a radial direction is referred to as a Y direction, and a central axis direction is referred to as a Z direction (refer to FIG. 2). The Z direction is orthogonal to the X direction and the Y direction, and is generally a body axis direction of the subject H.

The X-ray source 3 includes an X-ray tube 31, an X-ray filter 32, and a bowtie filter 33. The X-ray tube 31 generates X-rays, and irradiates the subject H with the generated X-rays. The X-ray filter 32 adjusts the dose of the X-rays emitted from the X-ray tube 31. The bowtie filter 33 optimizes an exposure dose by increasing the dose near the center and reducing the dose around the periphery in order to minimize the exposure dose in a peripheral portion.

As shown in FIG. 2, the X-ray detector 4 is configured by arranging a plurality of detector modules 40 in an arc shape in the X direction. Each of the detector modules 40 includes a collimator 41, a semiconductor layer 42, and an application specific integrated circuit (ASIC) 43. In addition, a plurality (four in FIG. 1) of heaters 46 and a plurality (three in FIG. 1) of cooling fans 47 are attached to a housing of the X-ray detector 4.

The collimator 41 is disposed on an X-ray incident side of the semiconductor layer 42, and removes scattered rays by restricting an incident direction of the X-rays onto the semiconductor layer 42. The semiconductor layer 42 is formed of cadmium zinc telluride (CZT), cadmium telluride (CdTe), or the like, and converts the X-rays that have passed through the subject H and are incident on the semiconductor layer 42, into charges corresponding to photons and outputs the charges.

The ASIC 43 is disposed on a side of the semiconductor layer 42 opposite to the collimator 41. The ASIC 43 is a circuit element having a photon counting circuit 44. The photon counting circuit 44 counts the charges output from the semiconductor layer 42 as the number of photons, and outputs a counting signal. Note that electrodes for applying a high voltage to the semiconductor layer 42 are formed on an upper surface and a lower surface of the semiconductor layer 42. The semiconductor layer 42 is configured with a plurality of pixels by patterning the electrodes on the lower surface side of the semiconductor layer 42. The photon counting circuit 44 counts photons for each pixel, and outputs the counting signal.

In addition, a temperature sensor 45 that measures a temperature of the ASIC 43 and outputs a measured value is provided inside the ASIC 43. In the ASIC 43, the temperature is changed with a temperature change of the semiconductor layer 42 caused by the flow of the current in a case where photons are incident on the semiconductor layer 42. The temperature change of the ASIC 43 at the time of the X-rays incidence depends on the counting rate of the photons by the photon counting circuit 44.

The heater 46 is driven and controlled by the controller 7 to heat the plurality of detector modules 40 of the detector 4 and increase the temperature of the ASIC 43.

The cooling fan 47 is disposed to blow air from the Z direction to the plurality of detector modules 40. In addition, the plurality of cooling fans 47 are driven and controlled by the controller 7. Accordingly, the cooling fan 47 cools the ASIC 43 to decrease the temperature of the ASIC 43.

The controller 7 is composed of a processor such as a central processing unit (CPU). The controller 7 controls the operations of the X-ray source 3, the X-ray detector 4, the gantry 5, and the examination table 6. Specifically, the controller 7 controls the irradiation of the X-rays from the X-ray tube 31 of the X-ray source 3, the detection of the X-rays by the X-ray detector 4, the rotation of the rotation plate 52 of the gantry 5, and the movement of the examination table 6. In addition, the controller 7 acquires the counting signal output from the photon counting circuit 44 of the ASIC 43, and the measured value of the temperature output from the temperature sensor 45.

The controller 7 comprises an auto exposure control (AEC). The controller 7 automatically determines a tube current for the X-ray tube 31 based on, for example, a positioning image for positioning before imaging, by the AEC. Then, the controller 7 performs a drive control of the X-ray tube 31 to emit the X-rays according to the determined tube voltage.

In addition, in a case where it is necessary to increase a temperature of a detector module 40 based on the measured value of the temperature from the temperature sensor 45, the controller 7 drives the heater 46 to increase the temperature of the detector module 40. On the other hand, in a case where it is necessary to reduce the temperature of the detector module 40, the controller 7 drives the cooling fan 47 to cool the detector module 40.

A supply of power, a supply of a control signal, and extraction of data between the controller 7, the X-ray source 3, and the X-ray detector 4 are performed through a slip ring (not shown) provided between the X-ray source 3, the X-ray detector 4, and the rotation plate 52.

The image processing unit 8 is an image processing processor that generates a tomographic image (that is, a CT image) by performing reconstruction processing based on the counting signals acquired from each ASIC 43 by the controller 7. The image processing unit 8 may be configured as a part of the controller 7.

In addition, an input device 9, a display device 10, a storage device 11, and a communication device 12 are connected to the controller 7. The input device 9 is a device for an operator to input an operation instruction, and is composed of a keyboard, a mouse, and the like. The display device 10 is a display such as a liquid crystal display, and displays an operation screen, a tomographic image, and the like. The storage device 11 is a memory, a storage device, or the like, and stores a tomographic image, a program, various kinds of information, and the like.

The communication device 12 is a communication interface for performing communication with radiology information systems (RIS), picture archiving and communication systems (PACS), and the like. The communication device 12 performs transmission control in accordance with a communication protocol defined by various wired or wireless communication standards.

FIG. 3 is an enlarged view of a portion of the detector 4 in the CT apparatus according to the first embodiment that is attached to the rotation plate 52. In addition, in FIG. 3, the cooling fan 47 of the detector 4 is not shown. As shown in FIG. 3, the detector 4 is provided with three columnar leg portions 48 at intervals, and the detector 4 is attached to the rotation plate 52 with a gap between the detector 4 and the rotation plate 52 by the three leg portions 48. An insulating and heat insulating material such as glass epoxy is interposed between the leg portion 48 and the rotation plate 52.

The heat dissipation mechanism 57 according to the first embodiment that dissipates heat of the detector 4 to the rotation plate 52 is provided in the gap between the detector 4 and the rotation plate 52. The heat dissipation mechanism 57 in the first embodiment includes a thermally conductive material 60 and a spring 63. The thermally conductive material 60 is moved to a position at which the detector 4 and the rotation plate 52 are caused to be thermally conductive from a non-contact position with the rotation plate 52 or the detector 4 in a situation where the temperature of the detector 4 rises. For this purpose, the thermally conductive material 60 is attached to be insertable into and retractable from the gap between the detector 4 and the rotation plate 52.

FIG. 4 is a cross-sectional view taken along line I-I of FIG. 3. As shown in FIG. 4, the thermally conductive material 60 consists of a first member 61 and a second member 62 having a right-angled triangular prism shape. The first member 61 and the second member 62 are disposed to be slidable with each other in the gap between the detector 4 and the rotation plate 52, with a surface serving as a long side of a right triangular prism shown in FIG. 4 as a sliding surface 60A. The sliding surface 60A is inclined with respect to surfaces of the rotation plate 52 and the detector 4 facing each other. The first member 61 and the second member 62 consist of a thermally conductive material. The sliding surface 60A between the first member 61 and the second member 62 is processed to have a surface roughness such that the first member 61 and the second member 62 can slide on each other.

As the thermally conductive material, it is preferable to use a material in which, in a case where a thermal conductivity is measured by a laser flash method based on JIS R 1611:2010 under the conditions of room temperature (25° C.) in the atmosphere using a disk having a diameter of 10 mm and a thickness of 1 mm as a sample size, the thermal conductivity is preferably 200 (W/m·K) or more and more preferably more than 400 (W/m·K). Examples of such a material include aluminum and copper.

A surface of the first member 61 opposite to the sliding surface 60A is fixed to the rotation plate 52. In addition, the spring 63 is disposed along a radial direction of the rotation plate 52. A support portion 53 protrudes toward the detector 4 side on a radially inner side of the rotation plate 52, and one end of the spring 63 is fixed to the support portion 53. The other end of the spring 63 on a radially outer side of the rotation plate 52 is fixed to a surface of the rotation plate 52 on the radially inner side of the second member 62. In a state where the rotation plate 52 is not rotated, the second member 62 is located at an initial position away from the detector 4 as shown in FIG. 4.

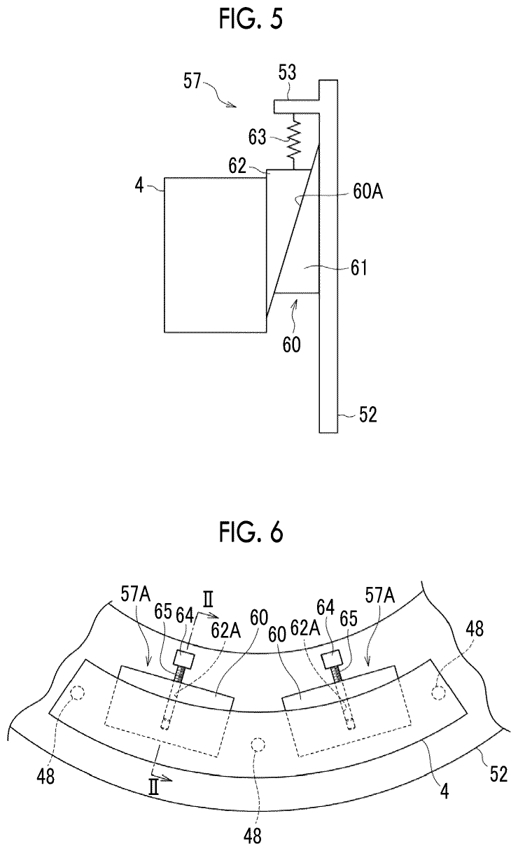

In a case where the subject H is imaged in the CT apparatus 2, the rotation plate 52 is rotated at a predetermined angular velocity. In a case where the rotation plate 52 is rotated, a centrifugal force acts on the second member 62, and the second member 62 moves outward from a rotation center of the rotation plate 52 against a spring force of the spring 63. As a result, as shown in FIG. 5, while the second member 62 maintains contact with the first member 61, a surface of the second member 62 opposite to the sliding surface 60A is in contact with the detector 4. A position of the second member 62 shown in FIG. 5 is set as an operation position.

A spring constant of the spring 63 is determined based on an angular velocity in a case where the rotation plate 52 is rotated, a position of the second member 62 in the radial direction of the rotation plate 52, and a weight of the second member 62 such that the second member 62 is located at the initial position shown in FIG. 4 in the state where the rotation plate 52 is not rotated and the second member 62 is moved to the operation position shown in FIG. 5 in a case where the rotation plate 52 is rotated. A position of the second member 62 in the radial direction of the rotation plate 52 is, for example, a position of a centroid of the second member 62 in the radial direction of the rotation plate 52.

In a case of imaging the subject H, the photons of the X-rays input to the semiconductor layer 42 of the detector module 40 are converted into electric charges, and the photon counting circuit 44 counts the converted electric charges. As a result, the detector module 40 generates heat. In this case, the temperature of the detector module 40 is measured by the temperature sensor 45, and in a case where the heat generation is insufficient, the heater 46 is driven to heat the detector module 40. On the other hand, in a case where the heat generation is large and the temperature measured by the temperature sensor 45 is equal to or higher than a predetermined threshold value Th0, the cooling fan 47 is driven to cool the detector module 40.

However, the heat generation of the photon counting circuit 44 may be too large, and the cooling may be insufficient only with the cooling fan 47. Such a lack of cooling occurs during the imaging of the subject H. The rotation plate 52 is rotated during imaging. In the first embodiment, in a case where the rotation plate 52 is rotated, a centrifugal force acts on the second member 62 of the thermally conductive material 60, and the second member 62 moves from the initial position shown in FIG. 4 to the operation position shown in FIG. 5 against the spring force of the spring 63. At the operation position, the detector 4 comes into contact with the rotation plate 52 through the thermally conductive material 60. Since the rotation plate 52 consists of metal and the thermally conductive material 60 consists of a thermally conductive material, the heat of the detector 4 is dissipated to the rotation plate 52 through the thermally conductive material 60.

As described above, in the first embodiment, in a situation where the detector 4 generates more heat, the detector 4 can be cooled more by the heat dissipation mechanism 57, and thus temperature stabilization of the detector 4 can be achieved.

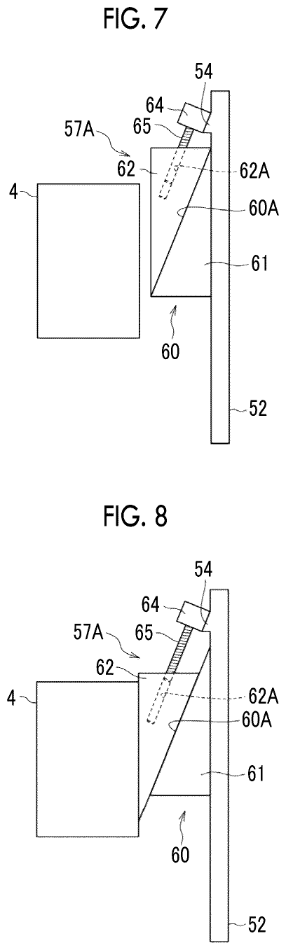

Next, a second embodiment of the present disclosure will be described. In the second embodiment, the same components as those in the first embodiment are assigned the same reference numerals, and a detailed description thereof will not be repeated here. FIG. 6 is a view showing a configuration of a heat dissipation mechanism in the CT apparatus 2 according to the second embodiment, and FIGS. 7 and 8 are cross-sectional views taken along line II-II in FIG. 6. As shown in FIG. 6, a heat dissipation mechanism 57A according to the second embodiment, which dissipates heat of the detector 4 to the rotation plate 52, is provided in the gap between the detector 4 and the rotation plate 52. The heat dissipation mechanism 57A in the second embodiment has the thermally conductive material 60 consisting of the first member 61 and the second member 62, similarly to the heat dissipation mechanism 57 in the first embodiment.

In the first embodiment, the second member 62 is moved against the spring 63 by the centrifugal force generated by the rotation of the rotation plate 52. In the second embodiment, the heat dissipation mechanism 57A includes a ball screw 65 that is screwed to a motor 64 and the second member 62, and the second member 62 is moved from the initial position in FIG. 7 to the operation position shown in FIG. 8 by rotating the ball screw 65 by the motor 64. The motor 64 and the ball screw 65 are an example of a driving unit of the present disclosure.

A support portion 54 protrudes toward the detector 4 side on the radially inner side of the rotation plate 52, and the motor 64 is attached to the support portion 54. One end of the ball screw 65 is attached to a rotation axis of the motor 64, and the other end thereof is screwed into a screw hole 62A formed in the second member 62. As shown in FIG. 7, in a case where the motor 64 is rotated in a predetermined direction in a state where the second member 62 is located at the initial position, the second member 62 is moved to the operation position as shown in FIG. 8. At the operation position, while the second member 62 maintains contact with the first member 61, a surface of the second member 62 opposite to the sliding surface 60A is in contact with the detector 4. In this state, the heat of the detector 4 is dissipated to the rotation plate 52 through the thermally conductive material 60. On the other hand, in a case where the motor 64 is rotated in a direction opposite to the predetermined direction in a state where the second member 62 is located at the operation position shown in FIG. 8, the second member 62 is moved to the initial position shown in FIG. 7.

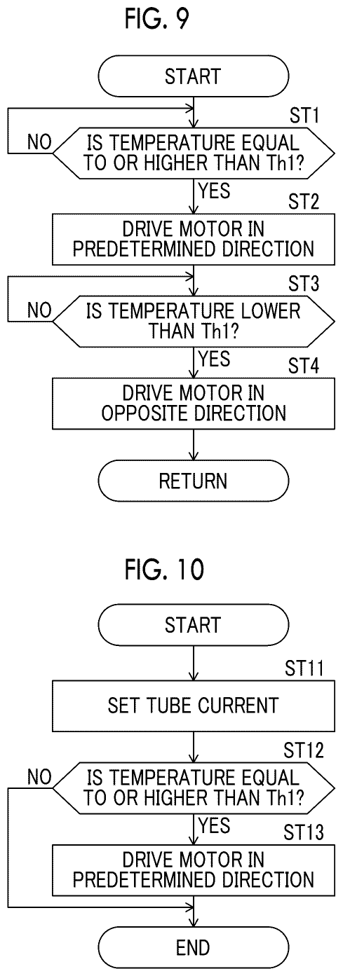

In the second embodiment, the rotation of the motor 64 is performed by a control signal from the controller 7 based on a measurement result of the temperature sensor 45. FIG. 9 is a flowchart showing processing performed by the controller 7 in the second embodiment. In the second embodiment, the controller 7 drives the heater 46 and the cooling fan 47 to perform the temperature stabilization of the detector module 40, but the description of the driving of the heater 46 and the cooling fan 47 will not be shown here.

As shown in FIG. 9, the controller 7 monitors whether or not the temperature of the detector module 40 detected by the temperature sensor 45 is equal to or higher than a predetermined threshold value Th1 (step ST1). The threshold value Th1 is, for example, a temperature at which the temperature stabilization of the detector module 40 cannot be maintained even in a case where the cooling fan 47 is driven, and is a temperature higher than the above-mentioned threshold value Th0. In a case where a positive determination is made in step ST1, the controller 7 drives the motor 64 to rotate in a predetermined direction (step ST2). As a result, the second member 62 is moved from the initial position to the operation position, and the temperature of the detector 4 is dissipated to the rotation plate 52 through the thermally conductive material 60.

Next, the controller 7 starts monitoring whether or not the temperature measured by the temperature sensor 45 is less than the threshold value Th1 (step ST3). In a case where a positive determination is made in step ST3, the controller 7 drives the motor 64 to rotate in a direction opposite to the predetermined direction (step ST4), and returns to step ST1. Accordingly, the second member 62 is moved from the operation position to the initial position. In this state, the temperature control by the heater 46 and the cooling fan 47 is performed.

As described above, in the second embodiment, in a case where the temperature of the detector module 40 is equal to or higher than the threshold value Th1, the motor 64 is driven in a predetermined direction to move the second member 62 from the initial position to the operation position. At the operation position, the detector 4 comes into contact with the rotation plate 52 through the thermally conductive material 60. Since the rotation plate 52 consists of metal and the thermally conductive material 60 consists of a thermally conductive material, the heat of the detector 4 is dissipated to the rotation plate 52 through the thermally conductive material 60. As described above, in the second embodiment, in a situation where the detector 4 generates more heat, the detector 4 can be cooled more by the heat dissipation mechanism 57, and thus temperature stabilization of the detector 4 can be achieved.

In the second embodiment, the motor 64 is driven to move the second member 62 from the initial position to the operation position based on the temperature detected by the temperature sensor 45, but the present invention is not limited thereto. The driving of the motor 64 may be controlled based on the irradiation conditions of the X-rays at the time of imaging. Hereinafter, this case will be described as a third embodiment. In the third embodiment, only the processing performed by the controller 7 is different from that of the second embodiment, and the configuration of the heat dissipation mechanism 57A is the same as that of the second embodiment. Therefore, the detailed description of the configuration will be shown.

FIG. 10 is a flowchart showing the processing performed in the third embodiment. It is assumed that the second member 62 is located at the initial position. As described above, since the heat generation of the photon counting circuit 44 is proportional to the number of photons of X-rays to be counted, the heat generation amount of the photon counting circuit 44 also increases as a tube current value of X-ray exposure increases. In the present embodiment, the controller 7 comprises the AEC and automatically sets the tube current in a case where the X-ray source 3 is driven (step ST11).

In the third embodiment, in a case where the subject H is imaged with the set tube current, the controller 7 determines whether or not the temperature of the photon counting circuit 44 is equal to or higher than the predetermined threshold value Th1 (step ST12). In a case where a positive determination is made in step ST12, the controller 7 drives the motor 64 in a predetermined direction (step ST13) to move the second member 62 from the initial position to the operation position and ends the processing of driving the heat dissipation mechanism 57A. In a case where a negative determination is made in step ST12, the controller 7 ends the processing of driving the heat dissipation mechanism 57A.

As described above, in the third embodiment, in a case where the tube current is set such that the temperature of the detector module 40 is equal to or higher than the threshold value Th1, the motor 64 is driven in a predetermined direction to move the second member 62 from the initial position to the operation position. At the operation position, the detector 4 comes into contact with the rotation plate 52 through the thermally conductive material 60. Since the rotation plate 52 consists of metal and the thermally conductive material 60 consists of a thermally conductive material, the heat of the detector 4 is dissipated to the rotation plate 52 through the thermally conductive material 60. As described above, in the second embodiment, in a situation where the detector 4 generates more heat, the detector 4 can be cooled more by the heat dissipation mechanism 57A, and thus temperature stabilization of the detector 4 can be achieved.

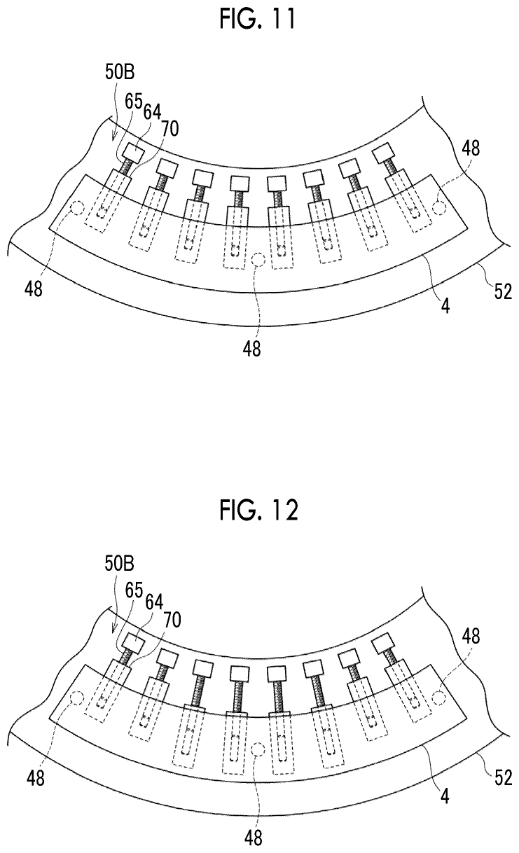

Next, a description regarding a fourth embodiment of the present disclosure will be made. In the fourth embodiment, the same components as those in the first embodiment are assigned the same reference numerals, and a detailed description thereof will not be repeated here. FIG. 11 is a view showing a configuration of a heat dissipation mechanism in the CT apparatus 2 according to the fourth embodiment. As shown in FIG. 11, the fourth embodiment is different from the first embodiment in that a plurality of heat dissipation mechanisms 57B driven by the motor 64 and the ball screw 65 are provided as in the second embodiment.

In the fourth embodiment, the thermally conductive material included in the heat dissipation mechanism 57B is divided into a plurality of small members 70 in a radial shape with respect to the rotation axis of the rotation plate 52. The small member 70 consists of the first member 61 and the second member 62 as in the first to third embodiments, and each second member 62 of the small member 70 is moved between the initial position and the operation position by the motor 64 and the ball screw 65. In the fourth embodiment, the controller 7 individually moves the second members 62 of the plurality of small members 70 to the operation position, so that a heat dissipation position and a heat dissipation amount from the detector 4 to the rotation plate 52 can be changed.

Here, in the detector 4, the plurality of detector modules 40 are arranged in an arc shape in the X direction as shown in FIG. 2. On the other hand, at a time of imaging, an irradiation amount of the X-rays to the detector module 40 varies depending on a physique of the subject H, the imaging part, or the like. In a case where the irradiation amount of the X-rays is different, the detector module 40 having a relatively large irradiation amount of the X-rays has a relatively large heat generation amount as compared with that of the detector module 40 having a relatively small irradiation amount of the X-rays.

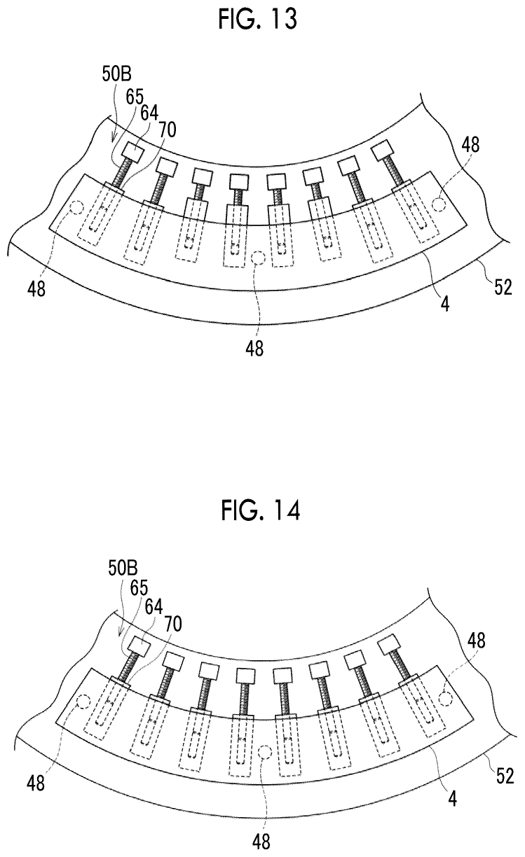

In the fourth embodiment, for example, in a case where the temperature of the detector module 40 near the center of the detector 4 is equal to or higher than the threshold value Th1, or in a case of an imaging condition in which an increase in the temperature of the detector module 40 near the center of the detector 4 is assumed, as shown in FIG. 12, the second member 62 of the small member 70 near the center of the detector 4 is moved from the initial position to the operation position. In addition, in a case where the irradiation range of the X-rays of the imaging part of the subject H is narrow such as the head or the limb, the temperature of the detector module 40 near an end part of the detector 4 increases or is assumed to increase. In such a case, as shown in FIG. 13, the second member 62 of the small member 70 near both end parts of the detector 4 is moved from the initial position to the operation position. In addition, in a case where the temperature of the detector module 40 is equal to or higher than the threshold value Th1 over the entire detector 4 or in a case of an imaging condition in which an increase in the temperature of the detector module 40 is assumed over the entire detector 4, as shown in FIG. 14, the second member 62 is moved from the initial position to the operation position for all the heat dissipation mechanisms 57B.

Accordingly, in the fourth embodiment, only the detector module 40 having a larger heat generation amount can be largely cooled in the detector 4. Therefore, the temperature stabilization of the detector 4 can be more efficiently achieved.

In each of the above-described embodiments, the first member 61 is fixed to the rotation plate 52, but the present invention is not limited thereto. The first member 61 may be fixed to the detector 4, and the second member 62 may be moved from the initial position to the operation position to bring the second member 62 into contact with the rotation plate 52 to dissipate the heat of the detector 4 to the rotation plate 52.

In each of the above-described embodiments, the temperature sensor 45 is provided inside each ASIC 43, but the temperature sensor 45 may be provided outside the ASIC 43. For example, a plurality of temperature sensors 45 may be disposed in a housing that accommodates the detector 4. It is preferable that the plurality of temperature sensors 45 are evenly arranged in the X direction and the Z direction. In this case, the controller 7 may acquire the measured value T of the temperature of each ASIC 43 from the temperature sensor 45 disposed in the vicinity of each ASIC 43.

In addition, in each of the above-described embodiments, the temperature sensor 45 is used to measure the temperature of the detector module 40, but the present invention is not limited thereto. The configuration may be set such that the overall temperature of the X-ray detector 4 such as a thermography camera can be measured.

In addition, in the second to fourth embodiments, the second member 62 is moved by the motor 64 and the ball screw 65, but the present invention is not limited thereto. A feed screw may be used instead of the ball screw 65. In addition, screws that mesh with each other may be provided on both the motor 64 and the second member 62, and the second member 62 may be moved by rotating the screw of the second member 62 by rotating the screw provided on the motor 64.

In addition, in each of the above-described embodiments, the X-rays have been described as an example of the radiation, but y-rays may be used as the radiation.

In addition, in the above-described embodiment, various processors described below can be used as the hardware structure of the controller 7.

The various processors include, in addition to a CPU that is a general-purpose processor that executes software (program) to function as various processing units, a programmable logic device (PLD) of which a circuit configuration can be changed after manufacturing, such as a field-programmable gate array (FPGA), and a dedicated electric circuit that is a processor having a circuit configuration dedicatedly designed for executing specific processing, such as an ASIC.

The various pieces of processing may be executed by one of the various processors or a combination of two or more processors of the same type or different types (for example, a plurality of FPGAs and a combination of CPU and FPGA). Further, a plurality of processing units may be configured with one processor. As an example where a plurality of processing units are composed of one processor, there is a form in which a processor that realizes all functions of a system including a plurality of processing units into one integrated circuit (IC) chip is used, such as a system on a chip (SOC).

Appendices of the present disclosure will be described below.

### Appendix 1

A radiography apparatus comprising:
a thermally conductive rotation plate that rotates about a rotation axis and to which a radiation source is fixed;
a radiation detector that includes a thermally conductive housing and is fixed to the rotation plate with a gap partially present between the radiation detector and the rotation plate at a position opposite to the radiation source across the rotation axis of the rotation plate; and
a heat dissipation mechanism that is disposed in the gap and has a thermally conductive material for dissipating heat of the radiation detector to the rotation plate,
wherein the thermally conductive material is moved to a position at which the radiation detector and the rotation plate are caused to be thermally conductive from a non-contact position with the rotation plate or the radiation detector in a situation where a temperature of the radiation detector rises.

### Appendix 2

The radiography apparatus according to Appendix 1,
wherein the thermally conductive material consists of a first member and a second member, each having a sliding surface that is inclined with respect to surfaces of the rotation plate and the radiation detector facing each other, and
the first member is fixed to the rotation plate or the radiation detector in the gap, the second member is located at a position away from the rotation plate or the radiation detector at an initial position, and the second member is moved to an operation position at which the second member comes into contact with the radiation detector or the rotation plate while sliding on the first member to maintain contact with the first member in the situation where the temperature of the radiation detector rises.

### Appendix 3

The radiography apparatus according to Appendix 2,
wherein the heat dissipation mechanism has a spring that is disposed along a radial direction of the rotation plate, of which one end is fixed to the rotation plate or the radiation detector and the other end located on a radially outer side of the rotation plate from the one end is fixed to the second member, and
a spring constant of the spring is determined based on an angular velocity in a case where the rotation plate rotates, a position of the second member in the radial direction of the rotation plate, and a weight of the second member.

### Appendix 4

The radiography apparatus according to Appendix 2,
wherein the heat dissipation mechanism has a driving unit including a motor and a ball screw of which one end is fixed to the motor and the other end is screwed to the second member, and
the driving unit moves the second member from the initial position to the operation position and moves the second member from the operation position to the initial position by driving the motor based on information related to a heat generation amount of the radiation detector.

### Appendix 5

The radiography apparatus according to Appendix 4,
wherein the information related to the heat generation amount is a temperature measured by a temperature sensor provided in the radiation detector.

### Appendix 6

The radiography apparatus according to Appendix 4,
wherein the information related to the heat generation amount is information based on a tube current applied to the radiation source.

### Appendix 7

The radiography apparatus according to any one of Appendices 4 to 6,
wherein the thermally conductive material is divided into a plurality of small members in a radial shape with respect to the rotation axis, and
the driving unit changes positions and the number of the small members that thermally conduct the radiation detector and the rotation plate according to at least one of a position at which the temperature in the radiation detector rises or the heat generation amount.

### Appendix 8

A temperature control method of a radiography apparatus including
a thermally conductive rotation plate that rotates about a rotation axis and to which a radiation source is fixed,
a radiation detector that includes a thermally conductive housing and is fixed to the rotation plate with a gap partially present between the radiation detector and the rotation plate at a position opposite to the radiation source across the rotation axis of the rotation plate, and
a heat dissipation mechanism that is disposed in the gap and has a thermally conductive material for dissipating heat of the radiation detector to the rotation plate, and a driving unit that moves the thermally conductive material to a position at which the radiation detector and the rotation plate are caused to be thermally conductive from a non-contact position with the rotation plate or the radiation detector in a situation where a temperature of the radiation detector rises, the method comprising:
acquiring information related to a heat generation amount of the radiation detector by a computer; and
controlling driving of the thermally conductive material by the driving unit based on the information related to the heat generation amount.

### Appendix 9

A temperature control program causing a computer to execute a temperature control method in a radiography apparatus including
a thermally conductive rotation plate that rotates about a rotation axis and to which a radiation source is fixed,
a radiation detector that includes a thermally conductive housing and is fixed to the rotation plate with a gap partially present between the radiation detector and the rotation plate at a position opposite to the radiation source across the rotation axis of the rotation plate, and
a heat dissipation mechanism that is disposed in the gap and has a thermally conductive material for dissipating heat of the radiation detector to the rotation plate, and a driving unit that moves the thermally conductive material to a position at which the radiation detector and the rotation plate are caused to be thermally conductive from a non-contact position with the rotation plate or the radiation detector in a situation where a temperature of the radiation detector rises, the program causing a computer to execute a process comprising:

acquiring information related to a heat generation amount of the radiation detector; and
controlling driving of the thermally conductive material by the driving unit based on the information related to the heat generation amount.

What is claimed is:

1. A radiography apparatus comprising:
a thermally conductive rotation plate that rotates about a rotation axis and to which a radiation source is fixed;
a radiation detector that includes a thermally conductive housing and is fixed to the rotation plate with a gap partially present between the radiation detector and the rotation plate at a position opposite to the radiation source across the rotation axis of the rotation plate; and
a heat dissipation mechanism that is disposed in the gap and has a thermally conductive material for dissipating heat of the radiation detector to the rotation plate,
wherein the thermally conductive material is moved to a position at which the radiation detector and the rotation plate are caused to be thermally conductive from a non-contact position with the rotation plate or the radiation detector in a situation where a temperature of the radiation detector rises.

2. The radiography apparatus according to claim 1,
wherein the thermally conductive material consists of a first member and a second member, each having a sliding surface that is inclined with respect to surfaces of the rotation plate and the radiation detector facing each other, and
the first member is fixed to the rotation plate or the radiation detector in the gap, the second member is located at a position away from the rotation plate or the radiation detector at an initial position, and the second member is moved to an operation position at which the second member comes into contact with the radiation detector or the rotation plate while sliding on the first member to maintain contact with the first member in the situation where the temperature of the radiation detector rises.

3. The radiography apparatus according to claim 2,
wherein the heat dissipation mechanism has a spring that is disposed along a radial direction of the rotation plate, of which one end is fixed to the rotation plate or the radiation detector and the other end located on a radially outer side of the rotation plate from the one end is fixed to the second member, and
a spring constant of the spring is determined based on an angular velocity in a case where the rotation plate rotates, a position of the second member in the radial direction of the rotation plate, and a weight of the second member.

4. The radiography apparatus according to claim 2,
wherein the heat dissipation mechanism has a driving unit including a motor and a ball screw of which one end is fixed to the motor and the other end is screwed to the second member, and
the driving unit moves the second member from the initial position to the operation position and moves the second member from the operation position to the initial position by driving the motor based on information related to a heat generation amount of the radiation detector.

5. The radiography apparatus according to claim 4,
wherein the information related to the heat generation amount is a temperature measured by a temperature sensor provided in the radiation detector.

6. The radiography apparatus according to claim 4,
wherein the information related to the heat generation amount is information based on a tube current applied to the radiation source.

7. The radiography apparatus according to claim 4,
wherein the thermally conductive material is divided into a plurality of small members in a radial shape with respect to the rotation axis, and
the driving unit changes positions and the number of the small members that thermally conduct the radiation detector and the rotation plate according to at least one of a position at which the temperature in the radiation detector rises or the heat generation amount.

8. A temperature control method of a radiography apparatus including
a thermally conductive rotation plate that rotates about a rotation axis and to which a radiation source is fixed,
a radiation detector that includes a thermally conductive housing and is fixed to the rotation plate with a gap partially present between the radiation detector and the rotation plate at a position opposite to the radiation source across the rotation axis of the rotation plate, and
a heat dissipation mechanism that is disposed in the gap and has a thermally conductive material for dissipating heat of the radiation detector to the rotation plate, and a driving unit that moves the thermally conductive material to a position at which the radiation detector and the rotation plate are caused to be thermally conductive from a non-contact position with the rotation plate or the radiation detector in a situation where a temperature of the radiation detector rises, the method comprising:
acquiring information related to a heat generation amount of the radiation detector by a computer; and
controlling driving of the thermally conductive material by the driving unit based on the information related to the heat generation amount.

9. A non-transitory computer-readable storage medium that stores a temperature control program causing a computer to execute a temperature control method in a radiography apparatus including
a thermally conductive rotation plate that rotates about a rotation axis and to which a radiation source is fixed,
a radiation detector that includes a thermally conductive housing and is fixed to the rotation plate with a gap partially present between the radiation detector and the rotation plate at a position opposite to the radiation source across the rotation axis of the rotation plate, and
a heat dissipation mechanism that is disposed in the gap and has a thermally conductive material for dissipating heat of the radiation detector to the rotation plate, and a driving unit that moves the thermally conductive material to a position at which the radiation detector and the rotation plate are caused to be thermally conductive from a non-contact position with the rotation plate or the radiation detector in a situation where a temperature of the radiation detector rises, the program causing a computer to execute a process comprising:
acquiring information related to a heat generation amount of the radiation detector; and
controlling driving of the thermally conductive material by the driving unit based on the information related to the heat generation amount.
